(19) United States
(12) Patent Application Publication
(10) Pub. No.: US 2026/0234103 A1
(43) Pub. Date: Aug. 13, 2026

(54) POLYTHIOL COMPOSITIONS AND THEIR PROCESS OF PREPARATION

(71) Applicant: ARKEMA FRANCE, Puteaux (FR)

(72) Inventors: Pierre-Thomas SKOWRON, Lacq (FR); Sophie MENDES, Lacq (FR); Kévin TOURNEUR, Lacq (FR); Nicolas JULLIAN, Puteaux (FR)

(73) Assignee: ARKEMA FRANCE, Puteaux (FR)

(21) Appl. No.: 19/469,890

(22) PCT Filed: Mar. 29, 2024
(86) PCT No.: PCT/FR2024/050416

§ 371 Date: Sep. 26, 2025

(30) Foreign Application Priority Data
Mar. 31, 2023 (FR) 2303201

(51) Int. Cl. C07C 319/12; C07C 67/16; C07C 319/28; C07D 251/34

(52) U.S. Cl. CPC C07C 319/12; C07C 67/16; C07D 251/34; C07C 319/28; C07C 2601/20

(57) ABSTRACT

The present invention relates to a process for the preparation of a polythiol from a polyene, and also to polythiol compositions obtained from cycloaliphatic polyenes or isocyanurate-type polyenes.

The process for the preparation of a polythiol comprises the following stages:
a) a polyene is reacted with a thiocarboxylic acid in the presence of oxygen (O₂) and of at least one organic solvent, so as to obtain a reaction medium comprising a polythioester and said at least one organic solvent;
b) a stage of deprotection of the polythioester obtained in stage a) is carried out, so as to obtain a polythiol;
in which stage a) and stage b) are carried out in one-pot synthesis.

The present invention relates to a process for the preparation of polythiol compositions, and also to the polythiol compositions obtainable by this process.

Polythiols are molecules of great industrial interest. They are used, for example, as crosslinking agents, in particular at low temperature.

There currently exist several synthetic routes for obtaining polythiols. Mention may be made, among the most widely used methods, of the reaction between polyols and mercaptoacids (described, for example, in Application US2005153231). Although this reaction is easy and makes it possible to obtain varied polythiol structures, the products obtained generally exhibit low resistances to hydrolysis because of the significant presence of ester functions.

Alternatively, the direct addition of hydrogen sulfide to polyenes by acid or photochemical catalysis makes it possible to obtain molecules without hydrolysable functions. This additive is described in particular in Application WO 12018757. However, with this method, large amounts of sulfide-type compounds can be co-produced and, depending on the reactants used, conversion problems can arise. Thus, the molecules obtained can comprise numerous unconverted double bonds, which generates stability problems and lowers the overall content of —SH functions. In the case of a starting reagent of triene type, for example, this is reflected in particular by the presence of mono- and/or dithiols in a large amount in the composition obtained.

In point of fact, the control of and/or the reduction in the formation of these by-products, such as mono- and/or dithiols, is important depending on the targeted fields of application. This is because their content has an influence on the degree of crosslinking of the subsequently prepared materials, in particular thermosetting materials produced from a resin and from a hardener of polythiol type. It has thus been demonstrated that the degree of crosslinking influences the physical and viscoelastic properties of the polymers, such as their density, their modulus, their limits of the elasticity range or their glass transition temperature (Tg). The Tg is conventionally determined by the DSC method for Differential Scanning calorimetry or by dynamic mechanical analysis (DM(T)A). These parameters are directly related to the behaviour of the materials, such as the hardness, the elasticity, the flexibility or also the tear strength.

In particular, it is sought to obtain polymers having a high glass transition temperature, in order to obtain materials with a higher thermal resistance (that is to say, which retain their characteristics over a wider temperature range).

There thus exists a need for a process for the industrial preparation of polythiols which makes it possible to control, indeed even to maximize, the conversion of the C═C double bonds into-SH functions. There also exists a need for a process for the preparation of polythiols which makes it possible to control, indeed even to reduce, the formation of by-products (for example mono- and/or dithiols in the case of the preparation of trithiols or also sulfides). One technical solution consists in passing through polythioester intermediates: the C═C double bonds are converted into functions of —R′—C(O)—S—R″ type, which are then deprotected in order to obtain the desired polythiols. However, these polythioester intermediates represent a great technical difficulty for industrial employment. They are generally very viscous, indeed even solid, compounds. They are thus difficult to be able to handle in order to be involved in the deprotection stage. They thus cause many practical problems at the industrial level and are in reality little used.

Moreover, these polythioesters are conventionally obtained by reaction of a polyene with a thiocarboxylic acid, in particular thioacetic acid. However, this reaction involves the use of a large excess of thiocarboxylic acid, which must be removed before the deprotection stage. Thus, additional stages of evaporation of this excess thiocarboxylic acid and/or of purification of the polythioesters are necessary in order to carry out the following deprotection stage. There thus exists a need for an improved process for the preparation of polythiols via polythioesters.

There exists a need for polythiol compositions, the content of —SH functions of which is controlled, indeed even maximized. The term “content of —SH functions” is understood to mean the ratio of the weight of all of the —SH functions/total weight of the composition.

It is an objective of the present invention to provide an improved process for the preparation of polythiol compositions, the industrial implementation of which is simplified.

It is an objective of the present invention to provide an improved process for the preparation of polythiol compositions by virtue of which the content of —SH functions is controlled, indeed even maximized.

It is also an objective of the present invention to provide improved polythiol compositions, in particular with a controlled, indeed even maximized, content of —SH functions.

In particular, it is an objective of the present invention to provide trithiol compositions with a controlled, indeed even reduced, amount of dithiols.

It is an objective of the present invention to provide polythiol compositions of use in the preparation of polymers, preferably of thermosetting polymers.

The present invention responds, in whole or in part, to the above objectives.

The present inventors have discovered, surprisingly, that it is possible to employ a “one-pot” process for the synthesis of polythiols. The term “one-pot process” is understood to mean in particular a process in which the synthesis intermediates (i.e. the polythioesters such as according to the invention) are not isolated from the reaction medium in order to carry out the following deprotection stage. In the context of the industrial synthesis of polythiols, such a one-pot process exhibits numerous advantages.

In particular, the stage of formation of the polythioester intermediates according to the invention (hereinafter stage a)) makes it possible to obtain a very good conversion (in particular between 90% and 100% conversion of the polyene) while avoiding the use of a too great excess of thiocarboxylic acid. In fact, a large excess of thiocarboxylic acid is conventionally used in the processes of the prior art, which represents a loss for the process and generates a large amount of waste to be isolated and treated. Moreover, such an excess is not compatible with a “one-pot” process because it is necessary to remove it before the deprotection stage. The present invention makes it possible to avoid these drawbacks, which represents an economic advantage but also an environmental advantage.

Another advantage of the present invention is that the reaction medium comprising the polythioester intermediates obtained on conclusion of stage a) can be stirred and handled easily. It can in particular exist in the form of a liquid or of a suspension, which is viscous or slightly viscous. Difficulties of operability at the industrial level are thus avoided. The reaction medium comprising the polythioester intermediates is also compatible with the deprotection stage (hereinafter stage b)), which represents a simplification of the process. Thus, stages a) and b) such as according to the invention are carried out as a “one-pot” reaction. The process is thus markedly improved because the intermediate stages of removal of the excess thiocarboxylic acid and/or of purification of the polythioester intermediates, such as extraction, recrystallization and/or distillation, are thus avoided.

The polythiol compositions capable of being obtained by the process according to the invention are novel and exhibit a controlled, indeed even maximized, —SH content. They are characterized in particular by a high

$$\frac{polythiol}{( x - 1 ) thiol ( s )}$$

ratio by weight as defined below. For example, in the case of a starting triene, said ratio by weight is the

$$\frac{trithiol}{dithiol ( s )}$$

ratio.

These compositions are particularly suitable for the preparation of materials such as thermosetting plastics, from resins. Thus, the present invention makes it possible to obtain materials with superior properties. For example, polymers having a higher Tg, thus a superior thermal resistance, and/or superior compressive strength properties and/or a greater modulus and a greater elasticity range, can be obtained.

Thus, the present invention relates to a process for the preparation of a polythiol comprising the following stages:
a) a polyene is reacted with a thiocarboxylic acid in the presence of oxygen (O₂) and of at least one organic solvent, so as to obtain a reaction medium comprising a polythioester and said at least one organic solvent; and
b) a stage of deprotection of the polythioester obtained in stage a) is carried out, so as to obtain a polythiol;
in which stage a) and stage b) are carried out in one-pot synthesis.

The present invention also relates to a polythiol composition A obtained from a cycloaliphatic polyene containing x C═C double bonds, said composition comprising:
the polythiol corresponding to said cycloaliphatic polyene comprising x-SH functions; and
the thiol(s) corresponding to said cycloaliphatic polyene comprising (x-1)—SH functions; and
in which the

$$\frac{polythiol}{( x - 1 ) thiol ( s )}$$

ratio by weight is of between 10.1:1 and 50,000:1, preferably between 10.1:1 and 20,000:1; and
x being an integer greater than or equal to 3.

The present invention relates to a polythiol composition B obtained from an isocyanurate of following general formula (I):

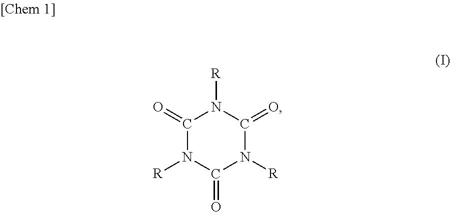

in which R is a linear or branched hydrocarbon chain comprising at least one C═C double bond, which can optionally comprise one or more heteroatom(s), such as oxygen, nitrogen, sulfur and/or phosphorus, and which can optionally comprise one or more chemical group(s); said composition comprising:
the polythiol corresponding to said isocyanurate comprising x-SH functions; and
the thiol(s) corresponding to said isocyanurate comprising (x-1)—SH functions; in which the

$$\frac{polythiol}{( x - 1 ) thiol ( s )}$$

ratio by weight is or between 2:1 and 50,000:1, preferably between 2:1 and 20,000:1; and
x being an integer greater than or equal to 3.

The term “alkyl” is understood to mean in particular a saturated, linear, branched or cyclic hydrocarbon radical comprising from 1 to 10, preferably from 1 to 4, carbon atoms. The term “aryl” is understood to mean in particular a cyclic (monocyclic, bicyclic or tricyclic) aromatic hydrocarbon radical comprising from 6 to 10 carbon atoms, preferably a phenyl or a naphthyl, more preferentially a phenyl.

The term “aralkyl” is understood to mean in particular an alkyl substituted by an aryl, for example benzyl.

### Polyenes

The term “polyene” is understood to mean any organic compound comprising at least 3 C═C double bonds. The number of C═C double bonds contained in said polyene is referred to hereinafter as “x”, x being an integer greater than or equal to 3. Preferably, x is between 3 and 10, more preferentially between 3 and 6, more preferably between 3 and 5.

Said polyene can be functionalized (i.e. comprise one or more chemical functions). Said polyene represents in particular a linear, branched, cyclic or branched cyclic hydrocarbon chain, which can optionally comprise one or more heteroatom(s), such as halogens, silicon, oxygen, nitrogen, sulfur and/or phosphorus, and which can optionally comprise one or more chemical group(s), for example chosen from halogens, —OH, —C(O)—, amine, amide, ester, ether, urea, thioether, sulfoxide, sulfone, carbamate or also thiocarbamate, preferably-OH or ether.

Such chemical groups are, for example, chosen from: —OH, —C(O)—, —NH₂, —NHR₈, —NR₈R₉, —C(O)NH₂, —C(O) NHR₈, —C(O) NR₈R₉, —C(O)OH, —C(O)OR₈, —NH—C(O)—NH₂, —NH—C(O)—NHR₈, —NH—C(O)—NR₈R₉, —NR₇—C(O)—NH₂, —NR₇—C(O)—NHR₈, —NR₇—C(O)—NR₈R₉, —NH—C(O)—OR₈, —NR₇—C(O)—OR₈, —O—C(═S)—NH₂, —O—C(═S)—NHR₈, —O—C(═S)—NR₈R₉, —S—C(═O)—NH₂, —S—C(═O)—NHR₈, —S—C(═O)—NR₈R₉, —S(═O)R₈ or —S(═O)₂R₈, and in which R₇, R₈ and R₉ are, independently of one another, chosen from the alkyls as defined above.

Said polyene can contain between 4 and 40, for example between 4 and 30, preferably between 4 and 20, more preferably between 10 and 15, carbon atoms. It is in particular aliphatic (i.e. non-aromatic).

Preferably, Said Polyene is Chosen from:
the cycloaliphatic polyenes and the polyenes of general formula (I) as defined below.
The cycloaliphatic polyenes

The term “cycloaliphatic polyene” is understood to mean non-aromatic cyclic polyenes. They can be cyclic and branched, as in the case of trivinylcyclohexane.

Preferably, they are not branched and their C═C double bonds are thus intracyclic. In particular, they are formed of a hydrocarbon chain preferably comprising between 7 and 15 carbon atoms.

Preferably, said cycloaliphatic polyene is 1,5,9-cyclododecatriene (1,5,9-CDT or CDT hereinafter).

### Mention May in Particular be Made of its Isomers, of Following Formulae:

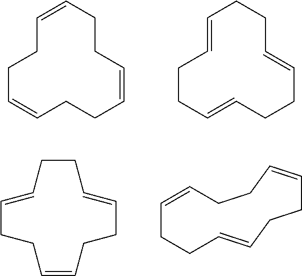

### Preference is Very Particularly Given to Cis, Trans, Trans-1,5,9-Cyclododecatriene, of Following Formula:

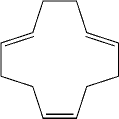 [SMILES: C1=C\CC/C=C/CC/C=C/CC/1]

The polyenes of isocyanurate type of following general formula (I):

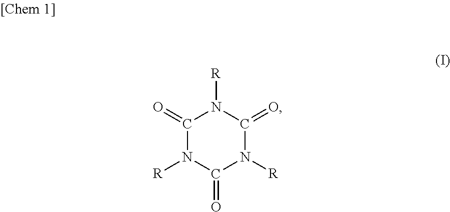

in which R is a linear or branched hydrocarbon chain comprising at least one C═C double bond, which can optionally comprise one or more heteroatom(s), such as oxygen, nitrogen, sulfur and/or phosphorus, and which can optionally comprise one or more chemical group(s) (in particular as mentioned above).

Preferably, R comprises a single C═C double bond and can optionally comprise one or more heteroatom(s) and in particular oxygen.

Preferably, R comprises between 2 and 15 carbon atoms, for example between 2 and 5 carbon atoms.

### The Following Two Compounds are Very Particularly Preferred:

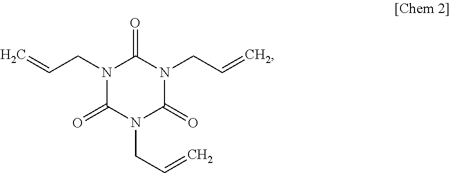 [SMILES: C=CCn1c(=O)n(CC=C)c(=O)n(CC=C)c1=O]

triallyl isocyanurate (hereinafter TAlC), and

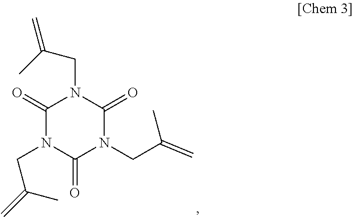 [SMILES: C=C(C)Cn1c(=O)n(CC(=C)C)c(=O)n(CC(=C)C)c1=O]

trimethallyl isocyanurate.
In particular, said polyene is chosen from CDT and TAlC.
Trienes

Preferably, said polyene is a triene. The term “triene” is understood to mean a polyene as defined above and comprising only 3 C═C double bonds (i.e. x=3).

It is Chosen in Particular from:
linear, branched or cycloaliphatic hydrocarbon trienes; and
the trienes of general formula (I) as defined above and in which R comprises a single C═C double bond.

### Mention May Specifically be Made, Among the Trienes, of:

trivinylcyclohexane, trivinylbenzene, cycloheptatriene, dimethylheptatriene, octatriene, cyclooctatriene, cyclododecatriene (CDT), triallyl isocyanurate (TAlC), triallyl cyanurate, trimethallyl isocyanurate, pentaerythritol triallyl ether, pentaerythritol tetraallyl ether, trimethylolpropane triallyl ether and triallylamine.

The preferred trienes according to the invention are cyclododecatriene (CDT) and triallyl isocyanurate (TAlC).

### Polythiols

According to the invention, the term “polythiol” refers to the polythiol corresponding to the starting polyene as defined above.

The term “corresponding to the starting polyene” is understood to mean that the structure of the starting polyene and of the polythiol obtained are identical, with the exception of the C═C double bonds, which have been converted into-SH functions (i.e. —CH—C(SH)—): for x C═C double bonds of the starting polyene, x-SH functions are obtained, with x as defined above.

Also included under the term “polythiol” are the polythiols which are positional isomers of the double bonds of the starting polyene. The polythiols according to the invention can also be referred to as (x)thiols.

### Polythioester Intermediates

The term “polythioester intermediate” or “polythioester” is understood to mean the polythioester corresponding to the starting polyene. The term “corresponding to the starting polyene” is understood to mean that the structure of the starting polyene and of the polythioester obtained are identical, with the exception of the C═C double bonds, which have been converted into —CH—C(S—C(O)—R₁)— functions (R₁ depends on the thiocarboxylic acid used; preferably, R₁ is a methyl): for x C═C double bonds, x thioester functions are obtained, with x as defined above. Also included under the term “polythioesters” are the polythioesters which are positional isomers of the double bonds of the starting polyene.

### Process According to the Invention

### Stage a)

During stage a), a polyene as defined above is reacted with a thiocarboxylic acid in the presence of oxygen (O₂) and of at least one organic solvent, so as to obtain a reaction medium comprising a polythioester as defined above and said at least one organic solvent.

The reaction is as follows: R—CH═CH—R+R₁—C(O)—SH→R—CH₂—CH(S—C(O)—R₁)—R
Stage a) is carried out in the presence of oxygen (O₂), acting here as initiator of the reaction.
Stage a) can thus be carried out in the presence of air, of depleted air (mixture of oxygen and nitrogen N₂) or of a mixture of oxygen and of another inert gas. The oxygen can be introduced into the reaction medium by any technique. The oxygen may or may not also be added throughout the duration of stage a).

In particular, the oxygen is bubbled into the reaction medium, preferably in the form of depleted air. For example, the depleted air is passed through a frit or a diffuser which dips into the reaction medium. Alternatively, oxygen can be bubbled into the reaction medium and nitrogen can be introduced into the gas phase of the reactor (that is to say, the headspace of the reactor).

The oxygen flow rate can be of between 0.01 and 100 Sl/h, preferably between 0.05 and 10 Sl/h, more preferably between 0.05 and 5 Sl/h, in particular between 0.05 and 2 Sl/h (standard litres/h).

Stage a) is carried out in particular in the absence of any other initiator of the reaction, and more preferentially in the absence of AIBN (azobisisobutyronitrile) and/or in the absence of UV radiation.

Stage a) is also carried out in the presence of an organic solvent or of a mixture of organic solvents. A polar solvent or a mixture of polar solvents is very particularly chosen. The solvent(s) can be polar protic or polar aprotic solvent(s). Mention may be made, among the solvents which can be used, of: alcohols, ethers (preferably cyclic ethers and glycol ethers, such as, for example, glycol dialkyl ethers), organochlorinated solvents, carboxylic acids or their mixtures.

### Preference is Given to Alcohols, in Particular of Following General Formula (IV):

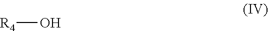

in which R₄ represents an alkyl as defined above. Preferably, the alcohol is chosen from the group consisting of: methanol, ethanol, isopropanol, n-propanol, n-butanol, butan-2-ol, isobutanol and tert-butanol, more preferably ethanol.

Preferably, the solvent is chosen from the group consisting of: tetrahydrofuran (THF), 2-methyltetrahydrofuran (Me-THF), dioxane, chloroform, acetic acid, methanol, ethanol, isopropanol, n-propanol, n-butanol, butan-2-ol, isobutanol, tert-butanol, dimethoxyethane (also referred to as glyme), diethoxyethane, dibutoxyethane and their mixtures, more preferentially ethanol.

The amount of solvent used is generally chosen as a function of the desired viscosity of the reaction medium. Complete or partial dissolution can be carried out by a person skilled in the art, depending on the targeted viscosity of the reaction medium. Preferentially, between 1 molar eq. and 50 molar eq., more preferably between 1 eq. and 20 eq., of solvent(s), with respect to the polyene, is (are) used.

The solvent can be added from the beginning of stage a), completely or partially. It can be added in a one-off manner in one go, in several goes (semi-continuously) or gradually (continuously), during stage a).

### The Thiocarboxylic Acid is Preferably of Following General Formula (II):

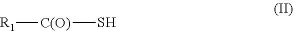

in which:
R₁ represents an alkyl radical, an aryl radical or an aralkyl radical as are defined above. Preferably, R₁ is chosen from methyl, ethyl and benzyl.
Thioacetic acid (here-below named TAA), for which R₁ is a methyl, is very particularly preferred according to the invention. For example, with thioacetic acid, a polythioacetate is obtained as polythioester intermediate.

According to one embodiment, the thiocarboxylic acid can be generated in situ (cf. the document U.S. Pat. No. 3,270,063, Thompson Chemical Co., 1963: “Methods of Making Primary Mercaptans”): thioacetic acid can be produced from acetic anhydride and hydrogen sulfide, in the presence of a catalyst.

Preferably, in order to carry out stage a), the thiocarboxylic acid and the solvent(s) are first introduced into the reactor, then the oxygen is introduced, for example by bubbling with air. The polyene can subsequently be added to the reaction medium.

The temperature of stage a) can be of between 5° C. and 80° C., preferably between 5° C. and 50° C., more particularly between 5° C. and 25° C., for example between 5° C. and 10° C. Stage a) is generally carried out at atmospheric pressure.

The thiocarboxylic acid/double bond of the polyene molar ratio can be of between 1 and 20, preferably between 1 and 10, for example between 1 and 5, more preferably between 1 and 3.

Stage a) makes it possible, starting from a polyene, to form a polythioester intermediate as defined above. The reaction medium obtained on conclusion of stage a) can thus comprise:
a polythioester intermediate as defined above;
the solvent or the mixture of solvents as defined above;
possibly by-products, such as (x-1) polythioesters; and
possibly one or more unreacted reactants.

The term “(x-1) polythioester” is understood to mean in particular a compound comprising x-1 thioester functions, with x being as defined above. It is a compound which has retained a C═C double bond (i.e. an unreacted C═C double bond).

The reaction medium can thus comprise between 10% and 85% by weight of polythioester intermediate, with respect to the whole of the reaction medium.

The reaction medium can comprise between 15% and 90% by weight of solvent(s), with respect to the whole of the reaction medium.

### Stage b)

Stage b) of deprotection of the polythioester intermediate obtained in stage a) makes it possible to obtain a polythiol. It can be carried out by any means known to a person skilled in the art. As stage a) and stage b) are carried out in a one-pot synthesis according to the invention, it is understood that the reaction medium comprising the polythioester obtained on conclusion of stage a) is retained in order to carry out the deprotection stage b). Thus, stages a) and b) are carried out in the presence of the same solvent (or mixture of solvents). It is possible to add more of said solvent (or said mixture of solvents) during stage b). In particular, the process according to the invention does not comprise a stage of separation and/or of extraction and/or of washing of the (organic) phase which comprises the polythioester between stages a) and b). In particular, no intermediate stage of purification of the polythioester is carried out. More particularly, no stage of recrystallization and/or of distillation of the polythioester is carried out.

The deprotection b) can be carried out by conventional methods: using a base or an acid, a catalyst of Dy(OTf)₃ type (cf. Liang et al., Asian J. Org. Chem., 10.1002/ajoc.201700481) or a compound of quaternary ammonium cyanide salt type (cf. U.S. Pat. No. 7,173,156).

Preferably, the deprotection b) is a basic deprotection, preferably in the presence of an alcohol as defined above. It is generally carried out by addition of an alkaline hydroxide, preferably NaOH or KOH. The addition can be carried out dropwise.

The deprotection stage b) can also be an acidic deprotection, preferably in the presence of an alcohol as defined above. It can be carried out with hydrochloric acid, methanesulfonic acid or anhydrous methanesulfonic acid. When the deprotection is acidic, it is preferable to use an alcohol as defined above as solvent.

### Sulfonic Acid

The sulfonic acid is preferably an organosulfonic acid which is optionally anhydrous. The sulfonic acid can be of following general formula (III):

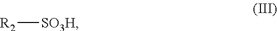

### Where R₂ Represents:

an alkyl radical, preferably as defined above, optionally substituted, in all or in part, by one or more identical or different halogen atoms, or
an aryl radical, preferably as defined above, optionally substituted by a saturated, linear or branched hydrocarbon chain comprising from 1 to 4 carbon atoms.

The halogen atom can be chosen from fluorine, chlorine and bromine. In particular, said alkyl can be perhalogenated, more particularly perfluorinated.

Preferably, the sulfonic acid is an alkanesulfonic acid which is optionally anhydrous (in the above formula, R₂ is an alkyl).

Thus, the Sulfonic Acids (and Also their Anhydrous Forms) can be Chosen from:
methanesulfonic acid, ethanesulfonic acid, n-propanesulfonic acid, isopropanesulfonic acid, n-butanesulfonic acid, isobutanesulfonic acid, sec-butanesulfonic acid, tert-butanesulfonic acid, trifluoromethanesulfonic acid, para-toluenesulfonic acid, benzenesulfonic acid and the mixtures of two or more of them in all proportions.

According to a very particularly preferred embodiment, the sulfonic acid used in the context of the present invention is methanesulfonic acid (MSA) or anhydrous methanesulfonic acid (AMSA).

Said sulfonic acid may or may not be supported. Preferably, it is not supported. When it is supported, it is possible, for example, to use sulfonated resins of styrene-divinylbenzene copolymer type, for example Amberlyst® 15 resin or Nafion®.

For example, between 0.1 and 10 eq. of acid are used for a polythioester.

For example, between 3 and 60 eq. (molar equivalent), preferably between 3 and 20 eq., of alcohol are used for a polythioester.

The deprotection stage b) can be carried out at a temperature of between 10° C. and 100° C., preferably between 25° C. and 80° C., more preferably between 40° C. and 80° C. It is generally carried out at atmospheric pressure.

Stages a) and b) can be carried out in the same reactor. For example, a batch reactor can be used.

Subsequent stages of conventional recovery and/or of conventional purification of the polythiol obtained on conclusion of stage b) can be carried out, depending on the desired degree of purity. For example, when the deprotection is carried out by the addition of a base, the reaction medium can subsequently be acidified and conversely, when the deprotection is carried out by the addition of an acid, the reaction medium can be basified. The organic phase resulting therefrom and comprising the various thiols (in particular the polythiol and the (x-1)thiols) can subsequently be extracted and optionally concentrated.

Thus and in particular, the polythiol obtained can be in the form of a polythiol composition as mentioned below.

### Compositions According to the Invention

When a polythiol is prepared from a polyene having x C═C double bonds, the conversion of these double bonds into-SH functions is not generally complete and by-products can be formed at each of the various stages, whatever the process used.

According to the invention, the term “polythiol” thus refers to the polythiol corresponding to the starting polyene and comprising x-SH functions. For example, trimercaptocyclododecane corresponds to cyclododecatriene. In this case, the conversion of the starting x C═C double bonds into-SH functions is complete.

According to the invention, the term “(x-1)thiol” refers to a thiol corresponding to the starting polyene and comprising (x-1)—SH functions. The term “corresponding to the starting polyene” is understood to mean that the structures of the starting polyene and of the (x-1)thiol obtained are identical, with the exception of the x C═C double bonds, which have been converted into (x-1)—SH functions. In this case, the conversion of the C═C double bonds into-SH functions has not been complete: an —SH function is missing. The C═C double bond not converted into an —SH function can in particular be:
still in the form of a C═C double bond; or
in the form of a thioester function which has not been deprotected.

There can thus exist different structures of (x-1)thiols, but they are here combined under this general name characterizing their number of —SH functions (unless specifically mentioned otherwise). Also included are the (x-1)thiols which are positional isomers of the double bonds of the starting polyene.

It is thus possible to obtain a polythiol composition resulting from a polyene having x C═C double bonds and comprising:
the polythiol corresponding to said polyene comprising x-SH functions; and
the thiol(s) corresponding to said polyene comprising (x-1)—SH functions.

Such a composition can optionally comprise other by-products or impurities (for example monothiols). It can be characterized by the

$$\frac{polythiol}{( x - 1 ) thiol ( s )}$$

ratio by weight. This is the ratio by weight: [polythiol corresponding to said polyene comprising x-SH functions]/[thiol(s) corresponding to said polyene comprising (x-1)—SH functions].
In Particular, a Trithiol Composition can be Obtained from a Triene, Said Composition Comprising:
the corresponding trithiol; and
the corresponding dithiol(s).

For example, in the case of a polythiol formed from cis, trans, trans-1,5,9-cyclododecatriene as starting polyene, the following dithioester can be obtained as by-product resulting from stage a):

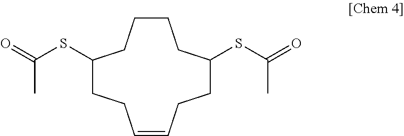 [SMILES: CC(=O)SC1CC/C=C\CCC(SC(C)=O)CCCC1]

During stage b), it is also possible for the deprotection not to be complete.

Thus, it is possible to form, according to the process according to the invention:
(x-1)thiols from the (x-1) thioesters formed in stage a); and/or
(x-1)thiols from the polythioesters which are not completely deprotected.

In the case of CDT, the following dithiols can thus be obtained on conclusion of stage b):

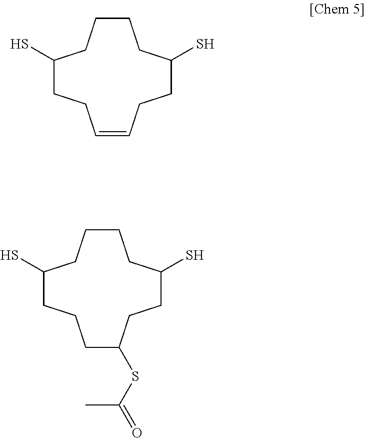 [SMILES: SC1CC/C=C\CCC(S)CCCC1 ; CC(=O)SC1CCCC(S)CCCCC(S)CC1]

Composition a Obtained from a Cycloaliphatic Polyene

The present invention relates to a polythiol composition A obtained from a cycloaliphatic polyene containing x C═C double bonds and as defined above, said composition comprising:
the polythiol corresponding to said cycloaliphatic polyene comprising x-SH functions; and
the thiol(s) corresponding to said cycloaliphatic polyene comprising (x-1)—SH functions (also referred to as (x-1)thiols as explained above),
   with x as defined above.

In particular, said composition A comprises at least 85% by weight, preferably at least 90% by weight, more preferably at least 95% by weight, of said polythiol, with respect to the total weight of the composition A.

In particular, said composition A comprises less than 9% by weight, preferably less than 5% by weight, more preferably less than 1% by weight, of said (x-1)thiol(s), with respect to the total weight of said composition A.

Preferably, the

$$\frac{polythiol}{( x - 1 ) thiol ( s )}$$

ratio by weight of the composition A is of between 10.1:1 and 20,000:1, preferably between 10.5:1 and 20,000:1, more preferably between 15:1 and 20,000:1, for example between 15:1 and 20,000:1.

Particularly preferably, the

$$\frac{polythiol}{( x - 1 ) thiol ( s )}$$

ratio by weight of the composition A is of between 10.1:1 and 10,000:1, preferably between 10.5:1 and 10,000:1, more preferably between 15:1 and 1000:1, for example between 15:1 and 500:1. More preferably still, the

$$\frac{polythiol}{( x - 1 ) thiol ( s )}$$

ratio by weight of the composition A is of between 10.5:1 and 1000:1.

The preferred starting cycloaliphatic polyene is cyclododecatriene, preferably 1,5,9-cyclododecatriene and more preferentially still its cis, trans, trans-1,5,9-cyclododecatriene isomer.

In particular, the present invention relates to a polythiol composition A obtained from cyclododecatriene as defined above, said composition comprising:
trimercaptocyclododecane;
dimercaptocyclododecene; and
S-[bis(sulfanyl)cyclododecyl]ethanethioate.

Thus, the [trimercaptocyclododecane/(dimercaptocyclododecene+S-[bis(sulfanyl)cyclododecyl]ethanethioate)] ratio by weight is in particular as defined above.

Composition B Obtained from a Polyene of Isocyanurate Type

The present invention also relates to a polythiol composition B obtained from an isocyanurate of following general formula (I) and as defined above:

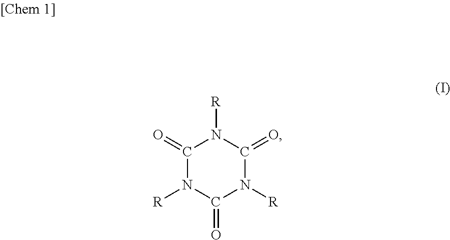

in which R is a linear or branched hydrocarbon chain comprising at least one C═C double bond, which can optionally comprise one or more heteroatom(s), such as oxygen, nitrogen, sulfur and/or phosphorus, and which can optionally comprise one or more chemical group(s); said composition comprising:
the polythiol corresponding to said isocyanurate comprising x-SH functions; and
the (x-1)thiol(s) corresponding to said isocyanurate comprising (x-1)—SH functions, with x as defined above.

In particular, said composition B comprises at least 70% by weight, preferably at least 80% by weight, more preferably at least 90% by weight, of said polythiol, for example at least 95% by weight of said polythiol, with respect to the total weight of the composition B.

In particular, said composition B comprises less than 30% by weight, preferably less than 20% by weight, more preferably less than 10% by weight, for example less than 5% by weight, indeed even less than 1% by weight, of said (x-1)thiol(s), with respect to the total weight of said composition B.

Preferably, the

$$\frac{polythiol}{( x - 1 ) thiol ( s )}$$

ratio by weight of the composition B is of between 2:1 and 20,000:1, for example between 2.3:1 and 20,000:1, preferably between 4:1 and 20,000:1, more preferably between 9:1 and 20,000:1, for example between 19:1 and 20,000:1.

Preferably, the

$$\frac{polythiol}{( x - 1 ) thiol ( s )}$$

ratio by weight of the composition B is of between 2:1 and 10,000:1, for example between 2.3:1 and 10,000:1, preferably between 4:1 and 10,000:1, more preferably between 9:1 and 1000:1, for example between 100:1 and 1000:1. More preferably still, the

$$\frac{polythiol}{( x - 1 ) thiol ( s )}$$

ratio by weight of the composition B is of between 10:1 and 1000:1.

The preferred starting isocyanurate is triallyl isocyanurate.

In particular, a polythiol composition B as defined above obtained from triallyl isocyanurate is obtained, said composition comprising:
1,3,5-tris(3-mercaptopropyl)-1,3,5-triazinane-2,4,6-trione and also its isomers; and
the corresponding dithiols (B1) and (B2) of following formulae and also their isomers:

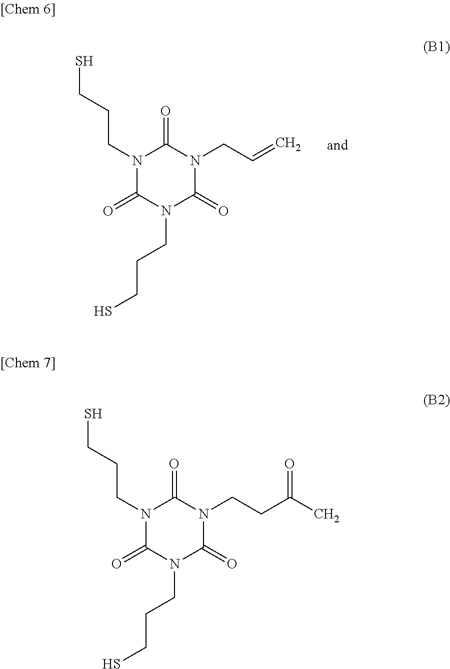 [SMILES: C=CCn1c(=O)n(CCCS)c(=O)n(CCCS)c1=O ; CC(=O)CCn1c(=O)n(CCCS)c(=O)n(CCCS)c1=O]

Thus, the [(1,3,5-tris(3-mercaptopropyl)-1,3,5-triazinane-2,4,6-trione)/(B1+B2)] ratio by weight is in particular as defined above.

The above compositions are novel and thus form part of the present invention.

The present invention also relates to said polythiol compositions capable of being obtained, obtained or directly obtained by the process according to the invention.

The present invention also relates to the polythiols obtained or directly obtained by the process according to the invention.

It is understood that, unless a specific isomer is mentioned, the name of a compound comprises all of its possible positional isomers.

It is understood that the ranges given, such as “of between X and X”, comprise the upper and lower limits.

The examples which follow are given by way of illustration and do not limit the present invention.

### EXAMPLES

### Example 1: One-Pot Synthesis of a Polythiol Composition from CDT According to the Invention, with Basic Deprotection

### Stage a):

155 g (2.04 mol) of TAA (thioacetic acid) are introduced into a 1-litre jacketed reactor. The medium is placed under stirring at 5° C. and then 28.4 g (0.62 mol) of ethanol are rapidly added. Air is bubbled into the reaction medium via a frit at a flow rate of approximately 0.4 Sl/h and nitrogen is passed into the headspace of the reactor at a flow rate of approximately 4 Sl/h.

100 g (0.62 mol) of 1,5,9-cyclododecatriene (CDT) are subsequently added dropwise via a peristaltic pump over approximately 1 h. Once the addition is finished, 113.6 g (2.47 mol) of EtOH are added to the reaction medium.

The reaction medium is kept stirred at 5° C. approximately overnight.

A GC/FID analysis shows complete conversion of the 1,5,9-cyclododecatriene. The air supply is cut off.

### Stage b) of Basic Deprotection:

171 g of EtOH are then added to the reaction medium. The reaction medium is subsequently degassed with nitrogen for 1 h, is then brought to 40° C. approximately and 177 g of a 46% aqueous sodium hydroxide solution, degassed beforehand, are added via a dropping funnel. The reaction medium is left stirring under nitrogen at 40° C. for 2 to 3 h.

### Recovery Stage:

The reaction medium is subsequently cooled to 20° C. and 200 g (2.03 mol) of 37% HCl are then added dropwise to the medium via a peristaltic pump. The organic phase comprising the thiols is withdrawn. The aqueous phase is extracted with 105 g (1.24 mol) of dichloromethane. The organic phases are subsequently combined, washed with 4 times with 14.8 g (0.82 mol) of water and then concentrated on a rotary evaporator.

A GC/FID analysis shows complete conversion of the trithioacetone.

### The Composition Obtained Comprises:

0.36% of the dithiol corresponding to CDT with 1 residual double bond,
0.47% of the dithiol monothioacetate corresponding to CDT,
95.87% of trimercaptocyclododecane, and
the remainder as impurities (100%).

$$\frac{polythiol}{( x - 1 ) thiol ( s )}$$

### Example 2: One-Pot Synthesis of a Polythiol Composition from CDT According to the Invention, with Acidic (AMSA and Ethanol) Deprotection

### Stage a):

155 g (2.04 mol) of TAA are introduced into a 1-litre jacketed reactor. The medium is placed under stirring at 5° C. and then 29 g (0.63 mol) of ethanol are rapidly added. Air is bubbled into the reaction medium via a frit at a flow rate of approximately 0.4 Sl/h and nitrogen is passed into the headspace of the reactor at a flow rate of approximately 4 Sl/h.

100 g (0.62 mol) of 1,5,9-cyclododecatriene are subsequently added dropwise via a peristaltic pump over approximately 1 h. Once the addition is finished, 113.6 g (2.47 mol) of EtOH are added to the reaction medium.

The reaction medium is kept stirred at 5° C. approximately overnight.

A GC/FID analysis shows complete conversion of the 1,5,9-cyclododecatriene. The air supply is cut off.

### Stage b) of Acidic Deprotection:

The temperature of the reaction medium is raised to 20° C. and 85 g (1.85 mol) of EtOH are added to the reaction medium. 74 g of anhydrous methanesulfonic acid (0.77 mol) are added dropwise via a dropping funnel. The medium is stirred at reflux under nitrogen for 10 h.

### Recovery Stage:

The reaction medium is subsequently cooled to 20° C. and 154 g (0.77 mol) of a 20% NaOH solution, degassed beforehand, are then added dropwise to the medium via a peristaltic pump. The organic phase comprising the thiols is withdrawn. The aqueous phase is extracted with 105 g (1.24 mol) of dichloromethane.

The organic phases are combined, then washed 4 times with 14.8 g (0.82 mol) of water and then concentrated on a rotary evaporator.

A GC/FID analysis shows complete conversion of the trithioacetate.

### The Composition Obtained Comprises:

5.26% of the dithiol corresponding to CDT with 1 residual double bond,
0.58% of the dithiol monothioacetate corresponding to CDT,
90.60% of trimercaptocyclododecane, and
the remainder as impurities (100%).

The

$$\frac{polythiol}{( x - 1 ) thiol ( s )}$$

ratio by weight is 90.60:5.84, i.e. 16:1.

### Example 3: One-Pot Synthesis of a Polythiol Composition from TAlC According to the Invention with Basic Deprotection

### Stage a):

101 g (1.33 mol) of TAA are introduced into a 1-litre jacketed reactor. The medium is placed under stirring at 5° C. and then 92 g (2.00 mol) of EtOH are rapidly added. Air is bubbled into the reaction medium via a frit at a flow rate of approximately 0.4 Sl/h and nitrogen is passed into the headspace of the reactor at a flow rate of approximately 4 Sl/h.

100 g (0.40 mol) of triallyl isocyanurate (TAlC) dissolved in 19 g (0.41 mol) of EtOH are subsequently added dropwise via a peristaltic pump over approximately 40 min. Once the addition is finished, 148 g (3.21 mol) of EtOH are gradually added to the reaction medium. The reaction medium is kept stirred at 5° C. for approximately 6 h.

A GC/FID or HPLC/UV analysis shows complete conversion of the triallyl isocyanurate. The air supply is cut off.

### Stage b) of Basic Deprotection:

The reaction medium is subsequently degassed with nitrogen for 1 h and then 115.1 g of a 46% aqueous sodium hydroxide solution, degassed beforehand, are added dropwise via a dropping funnel. The reaction medium is left stirring under nitrogen at 25° C. for 5 h and is then cooled to 10° C. for approximately 17 h.

### Recovery Stage:

The reaction medium is subsequently cooled to 20° C. and 483 g (1.32 mol) of a 10% HCl solution are then added dropwise to the medium via a peristaltic pump. The organic phase comprising the thiols is withdrawn. The aqueous phase is extracted with three times 68 g (0.80 mol) of dichloromethane.

The organic phases are combined, then washed twice with 14.4 g (0.80 mol) of water and then concentrated on a rotary evaporator.

A GC/FID analysis shows complete conversion of the trithioacetate.

A polythiol composition comprising 95% by weight of trithiol corresponding to TAlC and about 0.2% by weight of dithiol corresponding to TAlC, with respect to the total weight of the composition, is obtained.

The

$$\frac{polythiol}{( x - 1 ) thiol ( s )}$$

ratio by weight is 95:0.2, i.e. 475:1.

### Example 4: One-Pot Synthesis of a Polythiol Composition from CDT According to the Invention, with Acidic (HCl and Methanol) Deprotection

### Stage a):

155 g (2.04 mol) of TAA are introduced into a 3-litre jacketed reactor. The medium is placed under stirring at 5° C. and then 19.8 g (0.62 mol) of MeOH are rapidly added. Air is bubbled into the reaction medium via a frit at a flow rate of approximately 0.4 Sl/h and nitrogen is passed into the headspace of the reactor at a flow rate of approximately 4 Sl/h.

100 g (0.62 mol) of 1,5,9-cyclododecatriene are subsequently added dropwise via a peristaltic pump over approximately 1 h. Once the addition is finished, 78.9 g (2.46 mol) of MeOH are added to the reaction medium.

The reaction medium is kept stirred at 5° C. approximately overnight.

A GC/FID analysis shows complete conversion of the 1,5,9-cyclododecatriene. The air supply is cut off.

### Stage b) of Acidic Deprotection:

The reaction medium is raised to 20° C. and 1119 g (34.93 mol) of MeOH are added to the reaction medium. The medium is heated to 40° C., 576.9 g (5.85 mol) of 37% HCl are added dropwise via a peristaltic pump and then the medium is heated at reflux under nitrogen for 48 h.

### Recovery Stage:

The reaction medium is subsequently cooled to 20° C., the organic phase comprising the thiols is withdrawn and 105 g (1.24 mol) of dichloromethane are subsequently added to the aqueous phase.

The organic phases are combined, then washed four times with 74 g (4.11 mol) of water and then concentrated on a rotary evaporator.

A GC/FID analysis shows complete conversion of the trithioacetate. A polythiol composition comprising 96.28% of trimercaptocyclododecane and 0.25% of dithiol(s) corresponding to CDT is obtained.

The

$$\frac{polythiol}{( x - 1 ) thiol ( s )}$$

ratio by weight is 96.28:0.25, i.e. 385:1.

### Example 5: One-Pot Synthesis of a Polythiol Composition from CDT According to the Invention, with Acidic (HCl and Ethanol) Deprotection

### Stage a):

774.0 g (10.17 mol) of TAA are introduced into a 3-litre jacketed reactor. The medium is placed under stirring at 5° C. and then 142.0 g (3.08 mol) of ethanol are rapidly added. Air is bubbled into the reaction medium via a frit at a flow rate of approximately 0.4 Sl/h and nitrogen is passed into the headspace of the reactor at a flow rate of approximately 4 Sl/h.

500 g (3.08 mol) of 1,5,9-cyclododecatriene are subsequently added dropwise via a peristaltic pump over approximately 4 h30. Once the addition is finished, 851.7 g (18.49 mol) of ethanol are added to the reaction medium.

The reaction medium is kept stirred at 5° C. approximately overnight.

A GC/FID analysis shows complete conversion of the 1,5,9-cyclododecatriene.

The air supply is cut off.

### Stage b) of Acidic Deprotection:

The reaction medium temperature is raised to 20° C. and 1478.6 g (30.81 mol) of EtOH 96% are added to the reaction medium. The medium is heated to 40° C., and 910.9 g (9.24 mol) of 37% HCl are added dropwise via a peristaltic pump and then the medium is heated at reflux under nitrogen for 22 h.

### Recovery Stage:

The reaction medium is subsequently cooled to 30° C., the organic phase comprising the thiols is withdrawn and 261.7 g (3.08 mol) of dichloromethane are subsequently added to it. The obtained organic phase is then washed four times with 110.9 g (6.16 mol) of water and then concentrated on a rotary evaporator.

A GC/FID analysis shows complete conversion of the trithioacetate. A polythiol composition comprising 94.84% of trimercaptocyclododecane and 5.17% of dithiol(s) corresponding to CDT is obtained.

The

$$\frac{polythiol}{( x - 1 ) thiol ( s )}$$

ratio by weight is 94.84:5.17, i.e. 18.34:1.

### Example 6: One-Pot Synthesis of a Polythiol Composition from TAlC According to the Invention, with Acidic (HCl and Ethanol) Deprotection

### Stage a):

302.3 g (3.97 mol) of TAA are introduced into a 3-litre jacketed reactor. The medium is placed under stirring at 5° C. and then 166.3 g (3.61 mol) of ethanol are rapidly added. Air is bubbled into the reaction medium via a frit at a flow rate of approximately 0.4 Sl/h and nitrogen is passed into the headspace of the reactor at a flow rate of approximately 4 Sl/h.

300 g (1.20 mol) of triallyl isocyanurate in solution in 27.7 g (0.60 mol) of ethanol are subsequently added dropwise via a peristaltic pump over approximately 1 h15. Once the addition is finished, 221.8 g (4.81 mol) of ethanol are progressively added to the reaction medium.

The reaction medium is kept stirred at 5° C. approximately during about 6 h. A GC/FID or HLPC/UV analysis shows complete conversion of the triallyl isocyanurate. The air supply is cut off.

### Stage b) of Acidic Deprotection:

The reaction medium temperature is raised to 20° C. and 462.1 g (9.63 mol) of EtOH 96% are added to the reaction medium. The medium is heated to 40° C., and 355.8 g (3.61 mol) of 37% HCl are added dropwise via a peristaltic pump. Then the medium is heated at reflux under nitrogen for 28 h.

### Recovery Stage:

The reaction medium is subsequently cooled to 30° C., the organic phase comprising the thiols is withdrawn and 102.2 g (1.20 mol) of dichloromethane are subsequently added to it. The obtained organic phase is then washed four times with 43 g (2.41 mol) of water and then concentrated on a rotary evaporator.

A HPLC/UV analysis shows complete conversion of the trithioacetate.

A polythiol composition comprising 95.16% by weight of the trithiol corresponding to TAlC and about 0.2% by weight of dithiol(s) corresponding to TAlC is obtained, relative to the total weight of the composition.

The

$$\frac{polythiol}{( x - 1 ) thiol ( s )}$$

ratio by weight is 95.16:0.2, i.e. 476:1.

### Example Z: Applicative Test

Two polythiol compositions derived from triallyl isocyanurate (TAlC) were prepared for an application test. The mass ratios of trithiol/dithiol as well as the —SH percentages of these compositions are given in the table below:

| | P1 | |
|---|---|---|
| | (polythiol composition | P2 |
| | according | (comparative polythiol |
| | to the invention) | composition) |
| Ratio by weight | 476:1 | 1.31:1 |
| trithiol/dithiol | | |
| % SH | 27.3% | 21.8% |

The polythiol composition P1 is the one obtained according to example 6.

The polythiol composition P2 was prepared using the same protocol as described in example 6 but using AIBN (azobisisobutyronitrile) as initiator instead of oxygen and heating during step a) at 65° C.

The polythiol compositions P1 and P2 were used as hardeners for an epoxy resin (Araldite® LY 556 from Huntsman), with a tertiary amine (DABCO® 33-LV from Sigma-Aldrich) as catalyst. The curing was carried out at room temperature (i.e., 25° C.) using a stoichiometric amount of thiol functions relative to the epoxy functions. The quantities used are summarized in the table below:

| | Composition C1 | Composition C2 |
|---|---|---|
| | for curing with P1 | for curing with P2 |
| Araldite ® LY 556 (g) | 10 | 10 |
| DABCO ® 33-LV (g) | 0.3 | 0.3 |
| P1 (g) | 6.45 | — |
| P2 (g) | — | 8.07 |

The mixtures were prepared in aluminum cups: the quantity of Araldite® LY 556, polythiol, and amine were weighed respectively. The whole was quickly mixed with a spatula. The compositions began to harden within a few minutes and were left at room temperature for 7 days. A rod from each hardened composition was taken and placed on the rectangular torsion assembly fixed on an ARES rheometer (Rheometer Scientific) to perform a Dynamic Mechanical Analysis (DMA). The samples underwent a sinusoidal deformation at a frequency of 1 Hz over a temperature range varying from −50 to 110° C. The glass transition temperature (Tg) was determined at the maximum of the mechanical loss factor (tan delta). This value as well as the value of the elastic modulus at the plateau were recorded and are summarized in the table below:

| | Composition C1 | Composition C2 |
|---|---|---|
| | cured with P1 | cured with P2 |
| Tg (° C.) at max of tan delta | 64 | 58 |
| Elastic modulus G′ at the | 2.3 10⁶ | 1.5 10⁶ |
| plateau (Pa) | | |

The use of a polythiol composition P1 according to the invention in an epoxy resin thus allows obtaining a more crosslinked and denser network. The resin thus obtained is notably characterized by a higher Tg and a higher plateau modulus.

1. Process for the preparation of a polythiol comprising the following stages:
a) a polyene is reacted with a thiocarboxylic acid in the presence of oxygen (O₂) and of at least one organic solvent, so as to obtain a reaction medium comprising a polythioester and said at least one organic solvent; and
b) a stage of deprotection of the polythioester obtained in stage a) is carried out, so as to obtain a polythiol;
in which stage a) and stage b) are carried out in one-pot synthesis.

2. Preparation process according to claim 1, in which stage b) of deprotection is a basic deprotection, preferably carried out by addition of an alkaline hydroxide.

3. Preparation process according to claim 1, in which stage b) of deprotection is an acidic deprotection, preferably in the presence of an alcohol.

4. Preparation process according to claim 1 any one of the preceding claims, in which said reaction medium comprises between 15% and 90% by weight of solvent(s) at the end of stage a), relative to the total of said reaction medium.

5. Preparation process according to claim 1, in which said organic solvent is chosen from the group consisting of: alcohols, ethers, organochlorinated solvents, carboxylic acids and their mixtures.

6. Preparation process according to claim 1, in which the organic solvent is chosen from the alcohols of following general formula (IV): 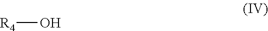
in which R₄ represents a saturated, linear, branched or cyclic hydrocarbon radical comprising from 1 to 10, preferably from 1 to 4, carbon atoms.

7. Preparation process according to claim 1, in which the thiocarboxylic acid is thioacetic acid.

8. Preparation process according to claim 1, in which said polyene is a triene.

9. Preparation process according to claim 1, in which the polyene is chosen from cycloaliphatic polyenes and isocyanurate polyenes of following general formula (I): 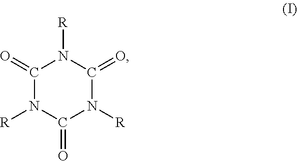
in which R is a linear or branched hydrocarbon chain comprising at least one C═C double bond, which can optionally comprise one or more heteroatom(s), such as oxygen, nitrogen, sulfur and/or phosphorus, and which can optionally comprise one or more chemical group(s).

10. Polythiol composition A obtained from a cycloaliphatic polyene containing x C═C double bonds, said composition comprising:
the polythiol corresponding to said cycloaliphatic polyene comprising x-SH functions; and
the thiol(s) corresponding to said cycloaliphatic polyene comprising (x-1)—SH functions; and
in which the
$$\frac{polythiol}{( x - 1 ) thiol ( s )}$$
ratio by weight is of between 10.1:1 and 50,000:1;
x being an integer greater than or equal to 3.

11. Polythiol composition A according to claim 10, in which said cycloaliphatic polyene is cyclododecatriene, preferably its cis, trans, trans-1,5,9-cyclododecatriene isomer.

12. Polythiol composition B obtained from an isocyanurate of following general formula (I): 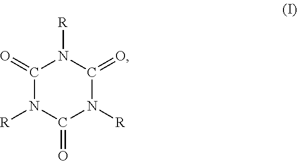
in which R is a linear or branched hydrocarbon chain comprising at least one C═C double bond, which can optionally comprise one or more heteroatom(s), such as oxygen, nitrogen, sulfur and/or phosphorus, and which can optionally comprise one or more chemical group(s);
said composition comprising:
the polythiol corresponding to said isocyanurate comprising x-SH functions; and
the thiol(s) corresponding to said isocyanurate comprising (x-1)—SH functions;
in which the
$$\frac{polythiol}{( x - 1 ) thiol ( s )}$$
ratio by weight is of between 2:1 and 50,000:1; and
x being an integer greater than or equal to 3.

13. Polythiol composition B according to claim 12, in which the isocyanurate is triallyl isocyanurate.
